(19) United States
(12) Patent Application Publication
(10) Pub. No.: US 2008/0227122 A1
(43) Pub. Date: Sep. 18, 2008

(54) P2Y PURINERGIC RECEPTOR EXPRESSION FOR IDENTIFYING PRENEOPLASTIC AND NEOPLASTIC STATES

(75) Inventors: Julian Barden, Marsfield (AU); Michael Slater, Belmore (AU)

Correspondence Address: TOWNSEND AND TOWNSEND AND CREW, LLP, TWO EMBARCADERO CENTER, EIGHTH FLOOR, SAN FRANCISCO, CA, 94111-3834, US

(73) Assignee: BIOSCEPTRE PTY LTD, Sydney (AU)

(21) Appl. No.: 12/043,083

(22) Filed: Mar. 5, 2008

Related U.S. Application Data
(63) Continuation of application No. 10/450,205, filed on Nov. 14, 2003, now abandoned.

(30) Foreign Application Priority Data
Dec. 11, 2000 (AU) PR 2015

(51) Int. Cl. G01N 33/574; C07K 16/30; C12N 5/06

(52) U.S. Cl. 435/7.23; 530/388.22; 530/389.7; 435/325

(57) ABSTRACT

The present invention relates to methods for identifying pre-neoplastic and neoplastic states in mammals and in particular to a method for identifying pre-neoplasia and neoplasia in cells and tissues based on differential expression of P2Y purineric receptors.

### CROSS-REFERENCES TO RELATED APPLICATIONS

This application is a continuation of U.S. application Ser. No. 10/450,205, filed Nov. 14, 2003, which is a U.S. National Phase of PCT/AU01/01614, filed Dec. 11, 2001, which claims priority to Australian Application No. PR 2015, filed Dec. 11, 2000, the entire contents of each of these applications are hereby incorporated herein by reference for all purposes.

### BACKGROUND OF THE INVENTION

1. Technical Field

The present invention relates to methods for identifying pre-neoplastic and neoplastic states in mammals and in particular to a method for identifying pre-neoplasia and neoplasia in cells and tissues based on differential expression of P2Y purinergic receptors.

2. Background

When diagnosing cancer, cellular features in biopsy samples are taken into account such as the degree of variability of cancer cell size and shape, the proportion of actively dividing cells and invasion into neighbouring structures. Commonly used histological stains are haematoxylin (primary stain) and eosin (counterstain) which differentially label subcellular elements. Other diagnostic methods employ antibodies to particular diagnostic molecules within (via intracellular epitopes) or on the surface of cells or tissues (via extracellular epitopes) which can be made visible for microscopic analysis eg, carcino-embryonic antigen (CEA). Some specific examples are discussed below.

### Prostate Cancer

The incidence of prostate cancer in the Western world is increasing at an alarming rate, having more than doubled in the past five years. It has the highest incidence of any neoplasm, is second only to lung cancer as the most common cause of cancer death in men worldwide, and is the leading cause of cancer death in Australia[1]. Benign prostatic hyperplasia (BHP) is common in men over 50 and is a possible precursor of prostatic intraepithelial neoplasia (PIN), itself a precursor to prostate cancer. Postmortem studies indicate that 70% of men have malignant cells in their prostate by the time they reach 80 [2].

Despite the gravity of this condition, diagnostic methods are few and imprecise. Current methods for assessing prognosis, such as digital rectal examination (DRE), ultrasound, prostatic acid phosphate levels, androgen ablation, prostate specific antigen (PSA) density, PSA velocity, PSA age-specific reference ranges and Gleason histopathological grading, can fail to provide reliable predictive information regarding the clinical outcome of prostate cancer [3]. For instance, studies have shown that DRE results in a 36.9% false negative rate [4]. PSA is a 33-kDa serine protease that is associated with a number of tissues besides prostate [5], is up-regulated by androgens, glucocorticoids and progestins and is thought to be involved in the regulation of growth factors. Unfortunately, serum PSA levels have an incidence of 23% false negative and 26.7% false positive diagnoses [5]. It has even been suggested that more than half of new screen-detected cases are in fact false positives [6]. Attempts to improve screening methods by the introduction of additional tests such as PSA density, velocity, and age-specific reference ranges has been equivocal. One study has shown that applying an age-specific PSA reference range that increases the upper limit of normal PSA to 4.5 ng/mL results in the failure to detect a substantial number of clinically significant cancers [7]. Given this uncertainty, prostate biopsy is often performed to confirm malignancy but this test also has a highly unsatisfactory 23% incidence of false-negative diagnosis [8], caused by the failure to directly sample the site of neoplastic change.

Treatment selection is largely dependent on clinical staging based on microscopic analysis of tissue sections [9]. This technique depends on judgment and considerable experience in relating histological appearance to clinical outcome. Unfortunately, prostate cancer tissue is notoriously heterogeneous and a vital diagnostic feature may easily be missed in the section being examined. To further complicate the situation, there have been no randomized and controlled trials to examine the outcomes of surgery and radiotherapy [2]. Treatment choices include radical prostatectomy, radiation therapy, androgen deprivation and “watchful waiting”. A definitive answer to the question of “watchful waiting” versus radical intervention awaits the conclusion of the prostate cancer intervention-versus-observation trial [10]. The consequences to the patient of these decisions are serious. Radical prostatectomy for instance, often results in incontinence, impotence, bladder neck stricture, pain and depression [11].

In the case of other cancers such as breast or skin, samples can be misdiagnosed for several reasons. IN the case of breast tumours, the borders of a localized tumour may not be well defined and similarly in the case of skin cancers, the borders between histologically obvious cancer (as determined with H&E stain) and normal tissue are variable. Either more or less normal tissue is excised around these tumours than is desireable.

It is an object of the present invention to provide a method of identifying pre-neoplastic and/or neoplastic cells which will overcome or substantially ameliorate at least some of the deficiencies of the prior art or will provide a useful alternative.

### BRIEF SUMMARY OF THE INVENTION

P2Y purinergic receptors are G-protein coupled tissue receptors which bind adenosine triphosphate (ATP) and, in the case of some P2Y receptor subtypes, uridine triphosphate (UTP). It has surprisingly been found that the expression pattern of P2Y receptors can be used to diagnose pre-neoplasia and neoplasia in mammals.

Pre-cancer and cancer stages in mammals are accompanied by marked differences in growth, extracellular matrix, metabolic and innervation factors as well as increases in subepithelial ionic calcium and microtubules. Using the novel antibodies of the present invention, the P2Y receptors can be really visualized with immunocytochemical methods and it has been shown that they present in a variety of expression patterns such as cell surface, tabular and punctuate labeling. These expression patterns can be used to identify the different stages in cancer development, commencing with entirely normal tissue and progressing through preneoplasia where P2Y receptor expression in the prostate as an example first appears in the nuclei within individual affected acini. In more advanced preneoplasia, but where there are still no morphological signs of disease the receptors leave the nuclei and spread into the cytoplasm and lateral and basal membranes within the acini prior to deposition on the apical membrane of the epithelial cells, an event occurring in synchrony with the morphological changes accompanying early cancer. Since these stages are more global in nature than the morphologically defined areas of obvious tumour, the diagnostic is not confined to direct sampling of the area of immediate tumour but can detect the tumour presence at some distance.

The invention therefore provides a new tool with which to diagnose pre-cancerous conditions (such as hyperplasia), to stage cancer and to investigate the basic physiology and aetiology of carcinogenesis.

According to a first aspect, the invention provides a method for diagnosing a pre-neoplastic and/or neoplastic state in a mammal, comprising detection of the P2Y purinergic receptor expression profile of a cell and/or tissue from said mammal and comparison of the profile with a predetermined expression profile of a normal cell and/or tissue.

According to a second aspect, the invention provides a method for staging a pre-neoplastic and/or neoplastic state in a mammal, comprising detection of the P2Y purinergic receptor expression profile of a cell and/or tissue from said mammal and comparison of the profile with a predetermined expression profile of a normal cell and/or tissue.

According to a third aspect, the invention provides a method of determining aetiology of carcinogenesis in a mammal, comprising detection of the P2Y purinergic receptor expression profile of a cell and/or tissue from the mammal and comparison of the profile with a predetermined expression profile of a normal cell and/or tissue.

Preferably, the mammal is a human but the method may be applied to any suitable mammal including a horse, a bovine animal, a sheep, a dog, a cat, a rat and a mouse.

Preferably, the cell and/or tissue is from a prostate, breast or skin. However, the skilled addressee will recognize that the method may also be useful for other cells types including other epithelial cells.

When the pre-neoplastic or neoplastic state is prostate pre-cancer or prostate cancer, an increase in the intensity of the P2Y₂ purinergic receptor expression profile in a prostate cell or prostate tissue, compared to the expression profile of a normal prostate cell or tissue, as herein defined, is diagnostic of the presence of prostate pre-cancer or prostate cancer.

When the pre-neoplastic or neoplastic state is skin pre-cancer or skin cancer, a decrease in the intensity of the P2Y₂ purinergic receptor expression profile in a skin cell and/or tissue from the mammal compared to the expression profile of a skin cell and/or tissue from normal skin, is diagnostic of the presence of skin pre-cancer or skin cancer.

Preferably, the skin cancer is malignant melanoma, squamous cell carcinoma or basal cell carcinoma. Thus, in accordance with the present invention, it is possible to differentiate skin cancer states, such as those mentioned, from keratoacanthoma and other benign lesions such as actinic keratoses.

Preferably, the P2Y receptor expression profile is the P2Y₂, P2Y₄ and/or P2Y₆ receptor expression profile and more preferably, it is the P2Y₂ receptor expression profile. The skilled addressee, without undue experimentation, can ascertain which P2Y receptors are most suitable for the particular method being practiced. Generally, those P2Y receptors which give the best contrast in terms of the difference in the intensity of expression profile between pre-neoplastic or neoplastic cells or tissues compared to normal cells or tissues are the most desirable.

In one embodiment, the pre-neoplastic state is an early pre-neoplastic state in which the P2Y receptor expression profile is altered in the nucleus of the cell. Alternatively, in another embodiment, the pre-neoplastic state is an advanced pre-neoplastic state in which the P2Y receptor expression profile is a profile in which the P2Y receptors are in the cytoplasm and/or lateral membranes and/or basal membranes.

In another embodiment, the neoplastic state is a state in which the P2Y receptor expression profile is a profile wherein the P2Y receptors are on the apical membrane of an epithelial cell.

It will be clear to the skilled addressee that the P2Y receptor expression profile can be combined with expression profile of other markers. In particular, the P2Y receptor expression profile can be combined with a P2X receptor expression profile and preferably, with the expression of P2X₁, and/or P2X₂.

Preferably, detection of the P2Y receptor expression profile is by immunohistochemical means. However, it will be clear to the skilled addressee that the P2Y receptors may be detected by other means including ELISA, RIA or similar immunological techniques, depending on the source of the cell or tissue sample and the reagents available. Preferably, the P2Y receptors are detected by a colorimetric assay such as DAB (diaminobenzidine) or the LSAB (labeled streptavidin biotin technique) secondary antibody detection kit (Dako).

It will be clear to those skilled in the art that standard Western blotting techniques and standard Northern detection of P2Y purinergic receptor mRNA may also be useful in determining the P2Y receptor expression profile.

According to a fourth aspect, the invention provides a P2Y purinergic receptor-specific antibody, wherein the antibody is capable of differentiating between pre-neoplastic or neoplastic cells and/or tissue and normal cells and/or tissue.

Preferably, the antibody is specific for P2Y₂, P2Y₄ and/or P2Y₆ receptors. Most preferably, the antibody is specific for P2Y₂ receptors.

It will be clear to those skilled in the art the P2Y receptor-specific antibodies may be polyclonal or monoclonal. It will also be clear to those skilled in the art that the P2Y receptor-specific antibody may be combined with other antibodies and, in particular, with one or more P2X receptor-specific antibodies. The P2X receptor-specific antibody may also be monoclonal or polyclonal. In a preferred embodiment, the P2X receptor antibody is a P2X₁ and/or P2X₂ receptor-specific antibody.

According to a fifth aspect, the present invention provides a suite of antibodies comprising antibodies according to the fourth aspect.

According to a sixth aspect, the invention provides a P2Y purinergic receptor-specific antibody according to the fourth aspect or a suite of antibodies according to the fifth aspect when used to differentiate between pre-neoplastic or neoplastic cells and/or tissue and normal cells and/or tissue.

According to a seventh aspect, the invention provides an isolated mammalian cell or tissue sample complexed with a P2Y purinergic receptor-specific antibody according to the fourth aspect or a suite of antibodies according to the fifth aspect.

The cells and/or tissue for use in the invention may be obtained by biopsy (for example, by fine needle aspirate, especially from breast tissue) but may also be obtained by any other means including from a body fluid, or in the case prostate cells and/or tissue, from digital rectal examination exudate or from semen.

It will be clear to the person skilled in the art that the methods and P2Y receptor-specific antibodies of the present invention may be used in combination with any other complementary test including, for example, a test for malignancy. The test for malignancy may be an anti-telomerase antibody test using a specific antibody against human telomerase protein 1(hTP₁) or any other suitable assay or test such as an anti-tenascin test.

According to a seventh aspect, the invention provides a kit for diagnosing a pre-neoplastic and/or neoplastic state in a mammal comprising means for detecting a P2Y purinergic receptor expression profile.

According to an eighth aspect, the present invention provides a kit for staging a pre-neoplastic and/or neoplastic state in a mammal comprising means for detecting a P2Y purinergic receptor expression profile.

According to a ninth aspect, the present invention provides a kit for determining the aetiology of carcinogenesis in a mammal comprising means for detecting a P2Y purinergic receptor expression profile.

Preferably, the means for detecting a P2Y purinergic receptor expression profile is an antibody according to the fourth aspect or a suite of antibodies according to the fifth aspect.

Preferably, the kit is used in a method according to any one of the first to third aspects.

In the context of the present invention, the term “neoplastic cell” and “neoplastic tissue” are to be construed in their ordinary sense and include, but are not limited to, a cell or tissue which has the characteristic feature of a neoplastic cell or tissue such as, for example, hyperplasia and/or a hypertrophic growth pattern and the characteristic staining with haematoxylin and eosin stain (ie. H&E) that highlights abnormal cell shape and size and nucleus size

In the context of the present invention, the terms “pre-neoplastic cell” and “pre-neoplastic tissue” are to be construed in their ordinary sense and include, but are not limited to, a cell or tissue that may be hyperplasic or hypertrophic. Such a cell or tissue, however, does not have the characteristic features of a neoplastic cell or tissue such as, for example, the characteristic staining with H&E that highlights abnormal cell shape and size and nucleus size.

In the context of the present invention, the terms “normal cell” and “normal tissue” are to be construed in their ordinary sense and include a cell or tissue in which the P2Y expression profile is not substantially altered from the profile that would be observed in the cells from an individual having neither pre-neoplasia nor neoplasia. In the context of the present invention as it relates to conditions of the prostate, the term “normal cell” also includes, for example, a cell from a prostate having benign prostate hyperplasia

In the context of the present invention the term “suite of antibodies” comprises polyclonal antibodies which contain several different antibodies specific for the same or different antigens and which are able to specifically differentiate between each of the receptor subtypes. When the antibodies are monoclonal, the term “suite of antibodies” also comprises a panel of antibodies able to specifically differentiate between each of the receptors e.g. P2Y and P2X or the receptor subtypes e.g. P2Y₂ and P2Y₄.

In the context of the present invention, detection of an “expression profile” comprises detection of a pattern or intensity of expression.

Unless the context clearly requires otherwise, throughout the description and the claims, the words ‘comprise’, ‘comprising’, and the like are to be construed in an inclusive sense as opposed to an exclusive or exhaustive sense; that is to say, in the sense of “including, but not limited to”.

### BRIEF DESCRIPTION OF THE DRAWINGS

FIG. 1 shows prominent epithelial nuclei labelled with P2Y₂ antibody, the first stage of preneoplasia.

FIG. 2 shows typical stage 2 staining of preneoplasia where the label now forms a punctate pattern (arrow) with no stain in the nuclei but equally with no morphological evidence yet of cancer.

FIG. 3 staining shows that P2Y₂ collects on the apical epithelium (arrow) in tissue showing evidence of morphological change associated with advanced cancer.

FIG. 4 shows a combination of P2Y₂, P2X₁ and P2X₂ antibody labelling of prostate tissue from a stage 3 case (obvious cancer).

FIG. 5 shows non-cancerous breast tissue (fibroadenoma) labelled with a combination of P2Y₂, P2X₁ and P2X₂ antibodies. A small improvement in label intensity is observed with the mixture of labels.

FIG. 6 shows a similar serial section of non-cancerous fibroadenoma labelled with P2Y₂ showing closely similar staining.

FIG. 7 shows cancerous breast tissue labelled with a combination of P2Y₂, P2X₁ and P2X₂ antibodies. The cancer tissue is devoid of label.

FIG. 8 shows breast cancer tissue labelled with P2Y₂ showing closely similar loss of positive staining.

FIG. 9 shows an example of a biopsy of a benign lesion in normal skin labelled with P2Y₂ antibody with prominent nuclei (PN) labelled.

FIG. 10 shows a biopsy section of malignant melanoma labelled with P2Y₂ antibody in which all the label that had been visible in the normal samples is lost in the melanoma. Normal label is regained within 2 mm of the melanoma. Combinations of antibodies used above yield identical results.

### DETAILED DESCRIPTION OF THE INVENTION

The present invention is based on the finding that pre-neoplastic and-neoplastic cells differentially express P2Y purinergic receptors. This differential expression can therefore be used to identify pre-neoplastic or neoplastic cells. Further, because the P2Y purinergic receptors are distributed differently in cells at different stages of pre-neoplasia and neoplasia, the expression profile of the P2Y purinergic receptors can be used to stage pre-neoplastic and neoplastic states. The differential expression patterns of P2Y can also be used to determine the aetiology of carcinogenesis. Further, the invention includes a kit for diagnosing a pre-neoplastic and/or neoplastic state comprising means for detecting a P2Y purinergic receptor expression profile; a kit for staging a pre-neoplastic and/or neoplastic state comprising means for detecting a P2Y purinergic receptor expression profile; and a kit for determining the aetiology of carcinogenesis comprising means for detecting a P2Y purinergic receptor expression profile.

Preferred embodiments of the invention will now be described by way of example only.

### EXAMPLE 1

### Antibody Production

The consensus sequences of the human P2Y₂[12], human P2Y₄[13], and human P2Y₆[14] cloned receptors were examined for suitable epitopes following the approach adopted in Hansen et al. [15]. The non-homologous epitope corresponding to the segment Leu226-Lys242 in P2Y₂ was used, with the comparable sequences in P2Y₄ (Arg226-Arg242) and P2Y₆ (Cys220-Lys236). Each was attached to diphtheria toxoid via maleimidocaproyl-N-hydroxysuccinimide crosslinker through adding an N-terminal Cys to P2Y₂ and P2Y₄ epitopes. All syntheses were carried out using standard t-BOC chemistry on an ABI synthesiser [16]. The peptide-antigen conjugates were suspended in water at 5 mg/mL and aliquots emulsified by mixing with Complete Freund's Adjuvant. Emulsion volumes of 1 mL containing 2 mg of peptide were injected intramuscularly with second, third, fourth and fifth immunisations followed at 2 week intervals using Incomplete Freund's Adjuvant. Final bleeds via venepuncture were obtained at 10-12 weeks, after it was established that adequate antibody titres had been obtained in the rabbits or sheep used for each epitope. The blood was incubated at 37° C. for 30 min, and stored at 4° C. for 15 h after which the serum was collected following centrifugation and stored at −20° C. in small aliquots. Sera were tested with an ELISA assay for antibodies specific for each peptide. The antibody titre, defined as the reciprocal of the serum dilution resulting in an absorbance of 1.0 above background in the ELISA assay, was in the range 75,000+−4,000 compared with 225+−25 for the pre-immune samples. Each antibody was found to be specific against the particular subtype.

Affinity purification of each of the antibodies against the specific epitope for that antibody resulted in reduced background but identical labelling trends. A monoclonal antibody raised against the same P2Y₂ epitope in mice yielded the same results.

### EXAMPLE 2

### Immunohistochemical Procedure

Sections with a thickness of 8 μm were cut from unfixed, frozen tissue using a Reichert Jung 2800 Frigocut cryotome or else were sectioned from paraffin-embedded tissue. Sections were air dried at room temperature for 1 hour, fixed for 12 hours in acetone at −20° C. and air dried at room temperature for 1 hour prior to antibody labelling. They were then incubated at room temperature with either a monoclonal mouse, rabbit or sheep anti-P2Y₂ primary antibody. After washing, sections were then incubated for 30 min in the secondary antibody: a 1:30 dilution of HRP-conjugated goat anti-mouse secondary antibody (Dako) for mouse primary, BRP-conjugated anti-rabbit secondary antibody for rabbit primary, or HRP-conjugated goat anti-sheep secondary antibody. Slides were again rinsed and then immersed in 15% diaminobenzidine tetrahydrochloride (DAB-Sigma) for 10 minutes. Sections were rinsed, air dried and mounted in DPX (Merck). Control slides were incubated in diluent buffer during the first incubation and then treated in the same manner as the experimental slides. Negative control slides were treated in the same manner as the experimental slides except that the primary antibody was replaced with non-immune serum.

Individual specificity of all antibodies was demonstrated.

### EXAMPLE 3

### P2Y Receptors in Human Cancer Tissue (Prostate)

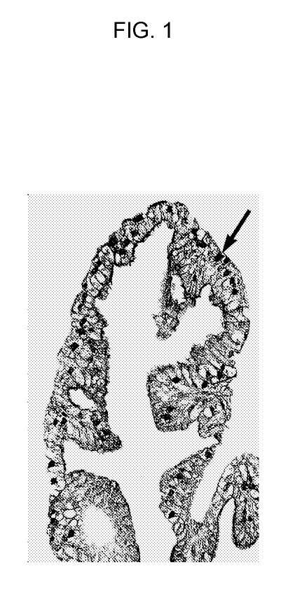

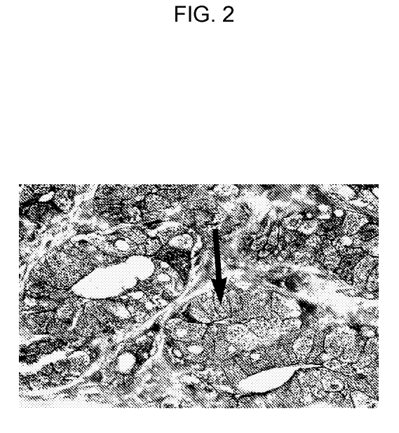

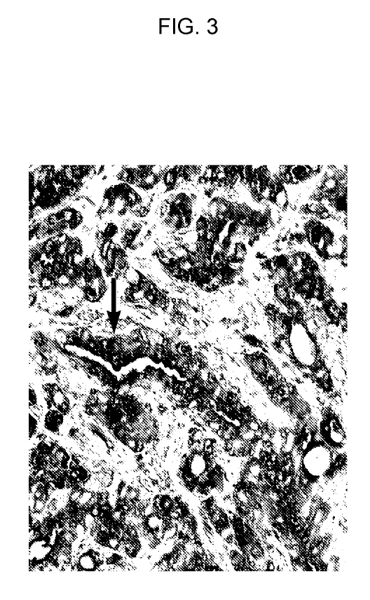

In a study of 40 normal and 40 human prostate cancer cases, P2Y₂ subtypes were markedly increased in human prostate cancer tissue. There was no observable labelling of normal tissue/benign prostatic hyperplasia. The labelling pattern for P2Y₂ in the cancerous tissue was particularly informative in that there was a greater proportion of labelled acinar epithelial cells with each stage of prostate disease, showing a direct correlation between neoplastic transformation and the extent of P2Y₂ acinar labelling commencing with the nuclei (stage 1; FIG. 1), progressing to extra-nuclear or cytoplasmic punctate labelling (stage 2; FIG. 2) and progressing to deposition on the apical epithelium and lateral epithelium (stage 3; FIG. 3) in advanced cases where stage 3 is also accompanied by changes in morphology.

### Combination of P2Y and P2X Receptor Antibodies

P2X are fast acting ligand (ATP)-gated ion channels that open the cell to ions, particularly calcium. P2Y receptors are much slower acting metabotropic receptors that act on internal cellular calcium stores following activation by ATP or UTP or even ADP by triggering a G-protein coupled cascade of activity and so function entirely differently to P2X receptors.

When P2X receptor antibodies were used in combination with P2Y receptor antibodies, a similar pattern to that described using P2Y receptor antibodies alone was obtained. In particular, labelling of prostate cells/tissues using antibodies against P2Y₂ and either or both P2X₁ and P2X₂ receptor subtypes enhanced results obtained using antibodies against the P2Y₂ receptor alone.

P2X antibodies were produced as follows:

The consensus sequences of the human P2X₁[17], human P2X₂[18], human P2X₃[19], human P2X₄[20], human P2X₅[21], human P2X₆[22] and human P2X₇[23] were examined for suitable epitopes following the approach used for the rat antibodies [15, 24]. The non-homologous epitopes corresponding to the segment Lys68-Val84 used in P2X₁ with an added N-terminal Cys. His209-Cys226 was selected from P2X₂. Asn185-Cys203 was selected in P2X₃. Cys270-Glu285 was selected from P2X₄. Cys272-Ser288 was selected from P2X₅. Asn200-Cys218 was selected from P2X₆. Val65-Lys81 to which was added a C-terminal Cys was selected from P2X₇. All epitopes were conjugated to diptheria toxin via maleimidocaproyl-N-hydroxysuccinimide for crosslinking to diphtheria toxin. Antibodies were raised in rabbits and sheep and purified as described above for the P2Y antibodies.

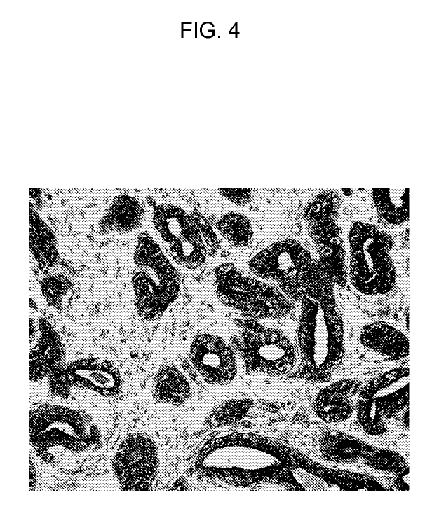

FIG. 4 shows the use of a combination of P2Y₂, P2X₁ and P2X₂ antibody labelling of prostate tissue from a stage 3 case (obvious cancer).

The use of other P2X subtypes such as P2X₃ and P2X₇ yielded similar results in prostate samples.

### EXAMPLE 4

### P2Y Receptors in Human Cancer Tissue (Breast and Skin)

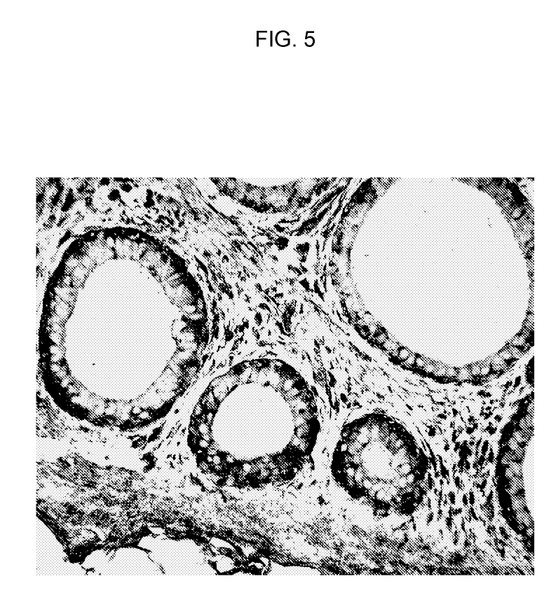

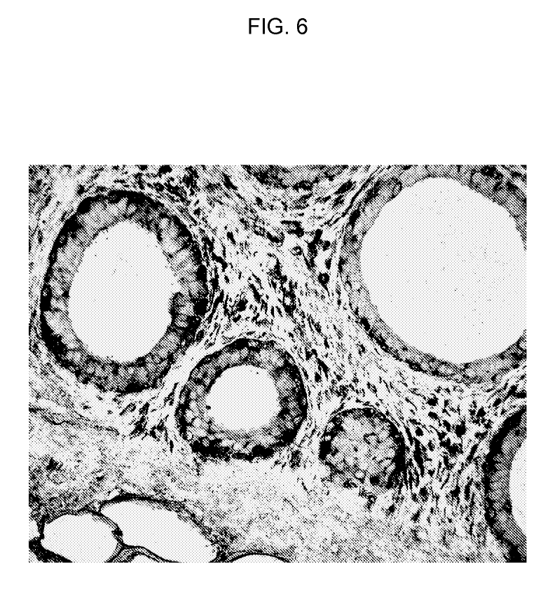

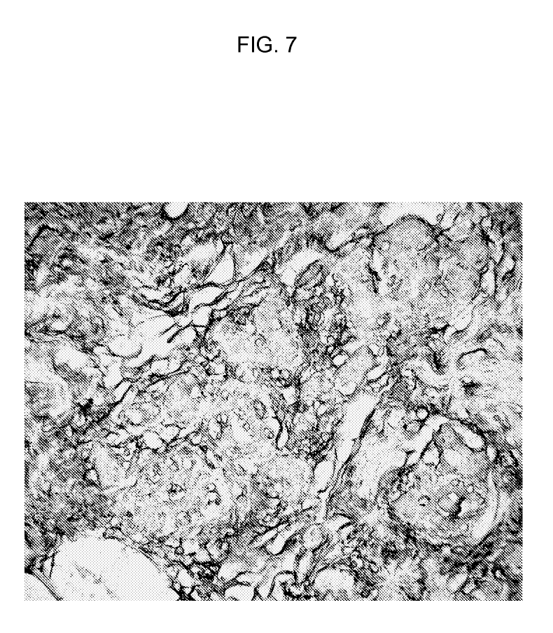

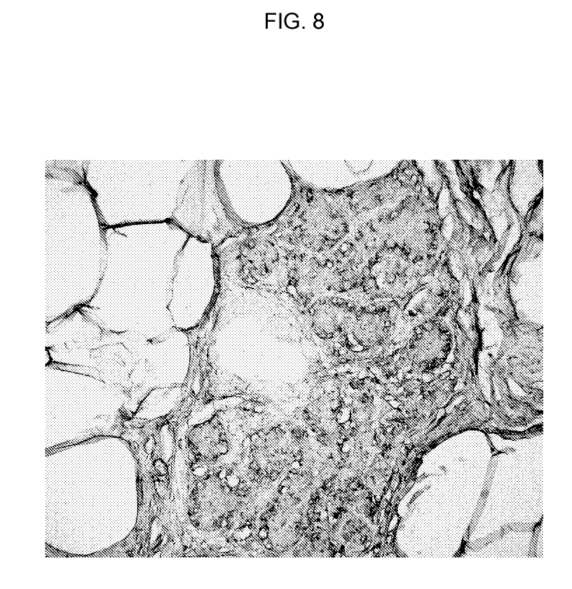

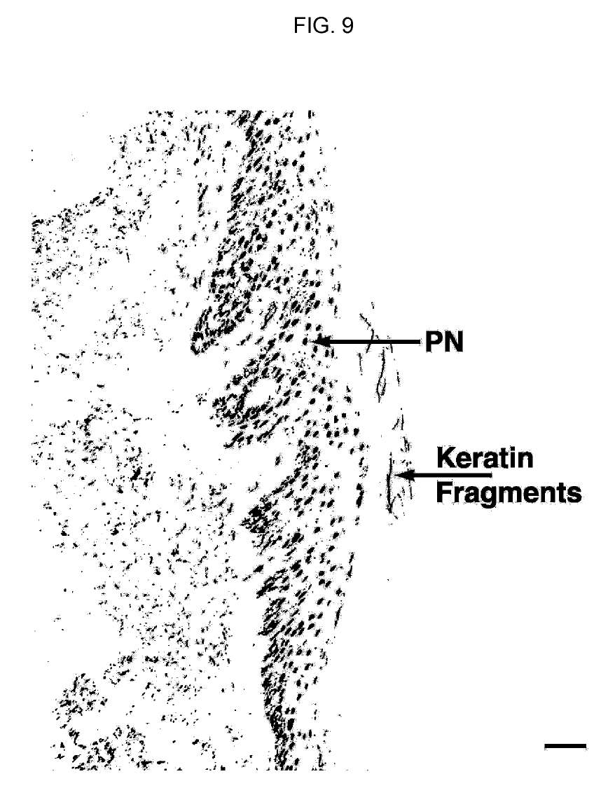

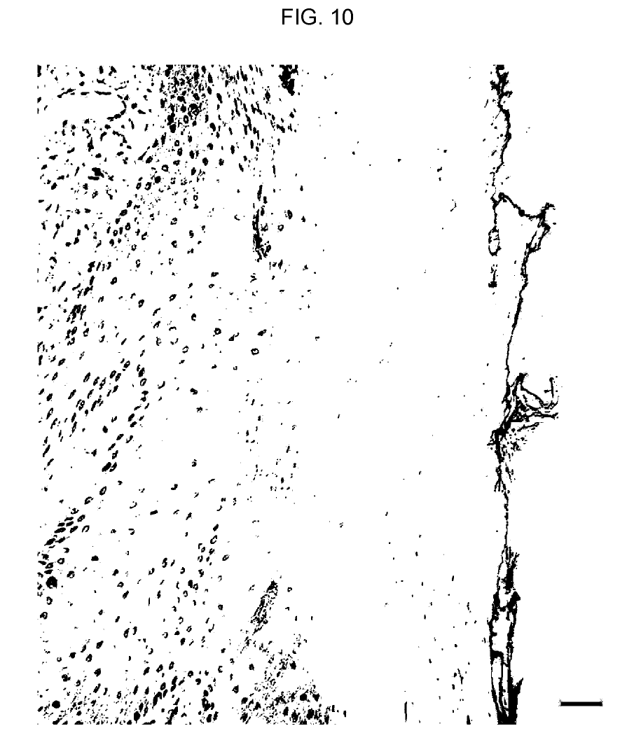

The labelling patterns were reversed in other cancers such as breast cancers (single and multi-foci) and skin cancers including malignant melanoma. In these tissues, P2Y₂ antibody labelling of normal tissue was intense (implying that the expression of P2Y₂ in normal tissue was relatively high) but P2Y₂ antibody labelling was not observed in cancer tissue. Results were enhanced by the use of antibodies against P2X receptors in combination with antibodies against P2Y₂ receptor, in particular antibodies against P2X₁ and P2X₂ subtypes (FIGS. 5 to 10).

### EXAMPLE 5

### Combining P2Y Receptor Antibody Diagnosis With Other Tests

It is clear that the P2Y (and P2X) receptor antibody diagnosis described above can be combined with complementary tests. Such tests may provide further information as to the nature of the cancer eg. a test for malignancy such as the anti-telomerase antibody test, and/or anti-tenascin may be useful in devising treatment regimes.

Although the invention has been described with reference to specific examples, it will be appreciated by those skilled in the art that the invention may be embodied in many other forms.

### REFERENCES

1. Lian F R, Bhuiyan M, Li Y W, Wall N, Kraut M, and Sarkar F H, 1998 Genistein-Induced G(2)-M Arrest, P21(Waf1) Upregulation, and Apoptosis in a Non-Small-Cell Lung Cancer Cell Line. Nutr. & Cancer. 31:184-191.

2. Hoey J, 1998 Prostate cancer: progress and perplexity. CMAJ 159:1-3.

3. Festuccia C, Vincentini C, di Pasquale A B, Aceto G, Zazzeroni F, Miano L, and Bologna M, 1995 Plasminogen activator activities in short-term tissue cultures of benign prostatic hyperplasia and prostatic carcinoma. Oncol. Res. 7:131-138.

4. Saxena S, Mohanty N K, and Jain A K, 1997 Screening of prostate cancer in males with prostatism. Ind. J. Pathol. Micro. 40:441-450.

5. Diamandis E P and Yu H, 1997 Nonprostatic sources of prostate-specific antigen. Urol. Clin. Nth. Am. 24:275-282.

6. Weyler J, 1999 Prostate cancer: screening or watchful waiting? Ann. Oncol. 9:9-11.

7. Bassler T J, Orozco R, Bassler I C, Odowd G J, and Stamey T A, 1998 Most Prostate Cancers Missed By Raising the Upper Limit of Normal Prostate-Specific Antigen For Men in Their Sixties Are Clinically Significant. Urol. 52:1064-1069.

8. Rabbani F, Stroumbakis N, Kava B R, Cookson M S, and Fair W R, 1998 Incidence and clinical significance of false-negative sextant prostate biopsies. J. Urol. 159:1247-1250.

9. Gao X, Porter A T, Grignon D J, Pontes J E, and Honn K V, 1997 Diagnostic and prognostic markers for human prostate cancer. Prostate 31:264-281.

10. Small E J, 1997 Prostate cancer. Curr. Opin. Oncol. 9:277-286.

11. Moul J W, Mooneyhan R M, Kao T C, McLeod D G, and Cruess D F, 1998 Preoperative and Operative Factors to Predict Incontinence, Impotence and Stricture After Radical Prostatectomy. Prost. Can. & Prost. Dis. 1:242-249.

12. Dasari V R, Sandhu A K, Mills D C, Athwal R S and Kunapuli S P, 1996 Mapping of the P2U purinergic receptor gene to human chromosome 11q 13.5-14.1 Somat. Cell Mol. Genet. 22, 75-79.

13. Communi D, Pirotton S, Parmentier M and Boeynaems J M, 1995 Cloning and functional expression of a human uridine nucleotide receptor J. Biol. Chem. 270, 30849-30852.

14. Maier R, Glatz A, Mosbacher J and Bilbe G, 1997 Cloning of P2Y6 cDNAs and identification of a pseudogene: comparison of P2Y receptor subtype expression in bone and brain tissues Biochem. Biophys. Res. Commun. 237, 297-302.

15. Hansen M A, Barden J A, Balcar V J, Keay K A, and Bennett M R, 1997 Structural motif and characteristics of the extracellular domain of P2X receptors. Biochem. Biophys. Res. Comm. 236:670-675.

16. Barden J A, Cuthbertson R M, Jia-Zhen W, Moseley J M, and Kemp B E, 1997 Solution structure of parathyroid hormone related protein (residues 1-34) containing an Ala substituted for an Ile in position 15 (PTHrP[Ala15]-(1-34)). J. Biol. Chem. 272:29572-29578.

17. Valera S, Talabot F, Evans R J, Gos A, Antonarakis S E, Morris M A and Buell G N, 1995 Characterization and chromosomal localization of a human P2X receptor from the urinary bladder. Recept. Channels 3, 283-289.

18. Lynch K J, Touma E, Niforatos W, Kage K L, Burgard E C, van Biesen T, Kowaluk E A and Jarvis M F, 1999 Molecular and Functional Characterization of Human P2X(2) Receptors. Mol. Pharmacol. 56, 1171-1181.

19. Garcia-Guzman M, Stuhmer W and Soto F, 1997 Molecular characterization and pharmacological properties of the human P2X3 purinoceptor Brain Res. Mol. Brain Res. 47, 59-66.

20. Garcia-Guzman M, Soto F, Gomez-Hemandez J M, Lund P E and Stubmer W, 1997 Characterization of recombinant human P2X4 receptor reveals pharmacological differences to the rat homologue. Mol. Pharmacol. 51, 109-118.

21. Le K T, Paquet M, Nouel D, Babinski K and Seguela P, 1997 Primary structure and expression of a naturally truncated human P2X ATP receptor subunit from brain and immune system. FEBS Lett. 418, 195-199.

22. Urano T, Nishimori H, Han H, Furuhata T, Kimura Y, Nakamura Y and Tokino T, 1997 Cloning of P2XM, a novel human P2X receptor gene regulated by p53 Cancer Res. 57, 3281-3287.

23. Buell G N, Talabot F, Gos A, Lorenz J, Lai E, Morris M A and Antonarakis S E, 1998 Gene structure and chromosomal localization of the human P2X7 receptor Recept. Channels 5, 347-354.

24. Dutton J, Hansen M, Balcar V, Barden J, and Bennett M R, 1999 Development of P2X receptor clusters on smooth muscle cells in relation to nerve varicosities in the rat urinary bladder. J. Neurocytol. 28, 3-15.

What is claimed is:

1. A method for diagnosing a pre-neoplastic and/or neoplastic state in a mammal, comprising detection of the P2Y purinergic receptor expression profile of a cell and/or tissue from said mammal and comparison of the profile with a predetermined expression profile of a normal cell and/or tissue.

2. A method for staging a pre-neoplastic and/or neoplastic state in a mammal, comprising detection of the P2Y purinergic receptor expression profile of a cell and/or tissue from said mammal and comparison of the profile with a predetermined expression profile of a normal cell and/or tissue.

3. A method according to claim 1 wherein the mammal is a human, a horse, a bovine animal, a sheep, a dog, a cat, a rat or a mouse.

4. A method according to claim 3 wherein the mammal is a human.

5. A method according to claim 1 wherein the cell and/or tissue is from a prostate, breast, skin or other epithelial source.

6. A method according to claim 1 wherein the pre-neoplastic or neoplastic state is prostate pre-cancer or prostate cancer and an increase in the intensity of the P2Y₂ purinergic receptor expression profile in a prostate cell or prostate tissue, compared to the expression profile of a normal prostate cell or tissue, as herein defined, is diagnostic of the presence of prostate pre-cancer or prostate cancer.

7. A method according to claim 1 wherein the pre-neoplastic or neoplastic state is breast pre-cancer or breast cancer and a decrease in the intensity of the P2Y₂ purinergic receptor expression profile in a breast cell and/or breast tissue obtained from the mammal compared to the expression profile of a breast cell and/or breast tissue from a normal breast, is diagnostic of the presence of breast pre-cancer or breast cancer.

8. A method according to claim 1 wherein the pre-neoplastic or neoplastic state is skin pre-cancer or skin cancer and a decrease in the intensity of the P2Y₂ purinergic receptor expression profile in a skin cell and/or tissue from the mammal compared to the expression profile of a skin cell and/or tissue from normal skin, is diagnostic of the presence of skin pre-cancer or skin cancer.

9. A method according to claim 8 wherein the skin cancer is malignant melanoma, squamous cell carcinoma or basal cell carcinoma.

10. A method according to claim 1 wherein the P2Y receptor expression profile is the P2Y₂, P2Y₄ and/or P2Y₆ receptor expression profile.

11. A method according to claim 10 wherein the P2Y receptor expression profile is the P2Y₂ receptor expression profile.

12. A method according to claim 1 wherein the pre-neoplastic state is an early pre-neoplastic state in which the P2Y receptor expression profile is altered in the nucleus of the cell.

13. A method according to claim 1 wherein the pre-neoplastic state is an advanced pre-neoplastic state in which the P2Y receptor expression profile is a profile in which the P2Y receptors are in the cytoplasm and/or lateral membranes and/or basal membranes.

14. A method according to claim 1 wherein the neoplastic state is a state in which the P2Y receptor expression profile is a profile wherein the P2Y receptors are on the apical membrane of an epithelial cell.

15. A method according to claim 1 wherein the P2Y receptor expression profile is combined with the expression profile of other markers.

16. A method according to claim 15 wherein the P2Y receptor expression profile is combined with a P2X receptor expression profile.

17. A method according to claim 16 wherein the P2Y receptor expression profile is combined with an expression profile of P2X₁ and/or P2X₂.

18. A method according to claim 1 wherein the P2Y receptor expression profile is detected by immunohistochemical means, or Western or Northern blotting techniques.

19. A method according to claim 1 wherein the P2Y receptor expression profile is detected by ELISA or RIA.

20. A method according to claim 1 wherein the P2Y receptor expression profile is detected by a colorimetric assay.

21. A P2Y purinergic receptor-specific antibody, wherein the antibody differentiates between a pre-neoplastic or neoplastic cell and/or tissue and a normal cell and/or tissue.

22. An antibody according to claim 21 wherein the antibody is specific for P2Y₂, P2Y₄ and/or P2Y₆ receptors.

23. An antibody according to claim 22 wherein the antibody is specific for P2Y₂ receptors.

24. An antibody according to claim 21 wherein the antibody is a monoclonal antibody.

25. An antibody according to claim 21 wherein the antibody is a polyclonal antibody.

26. An antibody according to claim 21 combined with at least one other antibody.

27. An antibody according to claim 26 wherein the at least one other antibody is a P2Y receptor-specific antibody or a P2X receptor-specific antibody.

28. An antibody according to claim 26 wherein the P2X receptor antibody is a P2X₁ or P2X₂ receptor-specific antibody.

29. A suite of P2Y antibodies comprising two or more different antibodies that differentiate between a pre-neoplastic or neoplastic cell and/or tissue and a normal cell and/or tissue.

30. A method for diagnosing a pre-neoplastic and/or neoplastic state in a mammal, comprising contacting the cell with an antibody of claim 21, detecting the P2Y purinergic receptor expression profile of the cell and/or tissue from the mammal, and comparing the profile with a predetermined expression profile of a normal cell and/or tissue.

31. A method for diagnosing a pre-neoplastic and/or neoplastic state in a mammal, comprising contacting the cell with a suite of antibodies of claim 29, detecting the P2Y purinergic receptor expression profile of the cell and/or tissue from the mammal, and comparing the profile with a predetermined expression profile of a normal cell and/or tissue.

32. A method according to claim 1 wherein the cell and/or tissue is obtained by biopsy.

33. A method according to claim 35 wherein the biopsy is taken by fine needle aspirate, from a body fluid, from digital rectal examination exudate or from semen.

34. A method according to claim 1 combined with a complementary test.

35. A method according to claim 34 wherein the complementary test is a test for malignancy.

36. A method according to claim 35 wherein the test for malignancy is an anti-telomerase antibody test using a specific antibody against human telomerase protein 1 (hTP₁) or an anti-tenascin test.

37. An isolated mammalian cell or tissue sample when used in a method according to claim 1.

38. A kit for diagnosing a pre-neoplastic and/or neoplastic state in a mammal comprising means for detecting a P2Y purinergic receptor expression profile.

39. A kit for staging a pre-neoplastic and/or neoplastic state in a mammal comprising means for detecting a P2Y purinergic receptor expression profile.

40. A kit according to claim 38 wherein the means for detecting a P2Y purinergic receptor expression profile comprises one or more P2Y purinergic receptor-specific antibodies, wherein the one or more antibodies differentiate between a pre-neoplastic or neoplastic cell and/or tissue and a normal cell and/or tissue.

41. A method according to claim 1 wherein the cell and/or tissue from said mammal is proximal to a pre-neoplastic or neoplastic cell and is not itself pre-neoplastic or neoplastic.
